(19) United States
(12) Patent Application Publication
(10) Pub. No.: US 2009/0099666 A1
(43) Pub. Date: Apr. 16, 2009

(54) Spongy implant

(75) Inventors: Markus Braun, Bochum (DE); Jurgen Speder, Bochum (DE); Martin Deli, Mulheim (DE); Volker Trosken, Witten (DE)

Correspondence Address: REED SMITH LLP, 3110 FAIRVIEW PARK DRIVE, SUITE 1400, FALLS CHURCH, VA, 22042, US

(73) Assignee: Amedo GMBH

(21) Appl. No.: 11/990,391

(22) PCT Filed: Aug. 9, 2006
(86) PCT No.: PCT/EP2006/007870

§ 371 Date: Nov. 5, 2008

(30) Foreign Application Priority Data
Aug. 13, 2005 (DE) 102005038381.5

(51) Int. Cl. A61F 2/02

(52) U.S. Cl. 623/23.72; 623/11.11

(57) ABSTRACT

The invention relates to the use of a spongy material for the production of an implant (10) designed to obstruct a cyst neck (3). As spongy material especially the polyvinyl alcohol Ivalon can be employed.

The invention furthermore relates to a set of instruments for the introduction of such an implant (10) made of a spongy material into a cyst neck (3) with said set of instruments consisting of a puncture needle (5) for the insertion of a guide wire (6), a guide wire (6) the distal end of which can be introduced into the cyst neck (3), an implant needle (7) provided at its proximal end with a connection element, a feeding unit (8) that can be attached via the connection element to the implant needle (7), and the implant (10) proper which consists of a spongy material and can be introduced via said feeding unit (8) through the implant needle (7) into the cyst neck (3). The inventive set of instruments may in particular be used for the treatment of Baker's cysts.

The invention relates to the use of a spongy material in the medical field. Moreover, the invention relates to a set of instruments for the manipulation of the material.

Cysts are single or multi-chamber saclike swellings closed off by a capsule and containing a liquid or semi-solid substance. For example, the so-called Baker's cyst, also termed popliteal cyst, is such a cyst which can be described as a fluid-filled sac located in the hollow of the knee. This cyst frequently arises through damage caused to the meniscus, for example a lesion of the medial meniscus or other chronic knee disease, such as for example osteoarthritis or chronic polyarthritis (rheumatism). The disease is accompanied with pain, swelling and restricted movement. Baker's cysts are frequently treated surgically by resection. Optionally, antiphlogistics, for example cortisone, may be administered by injection. Aside from this, it is known from DE 102 43 730 to use Linum usitatissimum seeds for the production of medical agents administered orally for the treatment of degenerative joint diseases. This is viewed as another way of treating irritated Bakers cysts.

However, with all the above described methods a high relapse rate is experienced, i.e. the recurrence of the disease after treatment has been completed. For that reason, there is demand for the provision of an apparatus and means which enable the treatment of cysts, in particular Bakers cysts, to be performed on a microinvasive and permanent basis. It is therefore the objective of the invention to provide means as well as an apparatus of this kind.

According to the invention this objective is reached by using a spongy material for the production of an implant by means of which a cyst neck is to be obstructed.

Cysts as the above described Baker's cysts have a neck via which the cyst is connected to a joint, for example with the knee joint in the case of a Bakers cyst. Through this cyst neck the joint fluid is allowed to penetrate into the tissue and in this manner lead to the cyst becoming filled. As provided for by the invention an implant is to be placed into the cyst neck and in this way obstruct the passage so that the joint fluid can no longer exit. For the production of this implant which is intended to plug up and thus obstruct the cyst neck a spongy material is to be used that on the one hand is sufficiently compressible and on the other capable of expanding to such an extent that it fills the cyst neck completely.

Basically, a variety of compound families may be used for the spongy material, for example polyvinyl alcohols (PVA), polyvinyl acetals, polyacrylates, polyurethanes, polystyrenes, polyvinyl oxides, polyvinyl fluorides, polyvinyl imidazoles, polyethylene oxides, polytetrafluoroethylene, an acyl-substituted cellulose acetate, a chlorine-sulfonated polyolefin or nylon. Especially preferred, however, is the use of a polyvinyl alcohol, in particular a polyvinyl alcohol known under the tradename of Ivalon. Ivalon is available from Unipoint Industries, High Point, N.C., USA. This is a material that cannot be decomposed biologically and is also employed for other medical applications. Ivalon is easily compressible and in this compressed form may be transferred by means of suitable cannulas to the target location where the material expands to fill up the cyst neck.

Since the implant shall fill the cyst neck completely it is expedient to adapt its shape to that of the cyst neck. In particular, the implant may thus be a cylindrically shaped element that is to be positioned such that its longitudinal axis extends along the longitudinal axis of the cyst neck. Additionally, cylindrical shape offers advantages in that the cylinder with its circular base area is capable of expanding uniformly in radial direction. Nevertheless, the cylinder may also have an oval base area.

It is, moreover, thought expedient to also match the length of the implant to that of the cyst neck to be filled so that the implant approximately is of equal length.

Aside from implants of cylindrical shape also cuboidal or ellipsoidal implants are conceivable. Such a cuboid may have a rectangular or square cross section; in the case of an ellipsoid an ellipsoid of revolution is to be given preference the cross section of which is circular and the longitudinal axis of which coincides, after positioning, with the longitudinal axis of the cyst neck. Such an ellipsoid is approximately equal in shape to that of a zeppelin or cigar.

The implant may also be provided additionally with thicker elements at its two longitudinal ends such that the shape of the implant roughly resembles a bone. These thicker additional elements may be used to additionally seal the cyst neck off at both ends.

With respect to the cross section as well the implant should be matched to the cyst neck. Therefore, to suit the relevant size an implant may used that in expanded state has a cross section, i.e. a diameter, ranging between 2 and 7 mm. Especially preferred are approx. 5 mm because this size is as a rule sufficient to completely fill up and effectively seal a cyst neck and on the other hand is still small enough to be inserted without difficulty.

Furthermore, it is considered expedient to design the implant in such a way that it can be visualized by image-forming methods. A suitable image-forming method is especially magnetic resonance (MR) imaging. Moreover, visualization may also be achieved by means of radiography. To enable visualization to be achieved the implant may be coated with (nano)particles. In the event MR images are to be produced these may be iron particles. If visualization is achieved by means of radiography palladium or platinum particles may be employed as well.

Aside from using a spongy material for the production of an implant designed to obstruct a cyst neck the invention also relates to a set of instruments by means of which such an implant can be introduced into the cyst neck. Such a set of instrument consists of a puncture needle for the insertion of a guide wire, a guide wire the distal end of which can be introduced into the cyst neck, an implant needle provided at its proximal era, i.e. the end located near the attending physician, with a connection element, a feeding unit that can be attached via the implant needle's connection element to the implant needle itself, and finally the implant proper which consists of a spongy material and can be introduced via said feeding unit through the implant needle into the cyst neck.

Such a set of instruments serves to enable the implant to be introduced into the cyst neck. For this purpose, the so-called Seldinger technique may be applied, a method named after the Swedish radiologist Sven-Ivar Seldinger. This procedure allows a catheter or similar devices to be inserted into a vessel without the need of operative intervention. At first the vessel, in this case the cyst, is punctured through the skin using a puncture (hollow) needle. Through the interior of the puncture needle a guide wire is advanced until its distal end enters the cyst neck. Through the puncture needle the fluid from the sac-like is cyst is now withdrawn. Following this, the cyst may be filled with a contrast medium to facilitate the visualization of the procedure through MR.

After this has been accomplished the puncture needle can be removed and an implant needle inserted into the cyst, with said implant needle being hollow so that it can be passed over the guide wire, i.e. the guide wire extends through the lumen of the implant needle. At the proximal end of the implant needle there is a connection element via which a feeding unit can be attached to the implant needle. The implant consisting of a spongy material, in particular Ivalon, can then be introduced into the cyst neck via the feeding unit and through the implant needle.

Preferably, the set of instruments may furthermore comprise a dilatator. Such a dilatator is arranged inside the implant needle or is introduced through it and serves to expand the cyst neck end near the cyst to such an extent that the implant can be more easily placed into the cyst neck in a safe and reliable manner.

The connection element located at the proximal end of the implant needle may in particular be the half of a Luer-Lock connection whereas the matching counterpart is arranged at the feeding unit. The so-called Luer system has proven its worth many times especially for application in the medical field. It may be described as a standardized conical connection system providing for a slightly tapered connector to be inserted into an opening arranged in the counterpart to effectively prevent the connection made up in this way from separating an additional screw-lock feature is incorporated resulting in the above mentioned Luer-Lock principle to be implemented. Accordingly, the male connector is also provided with an additional inner thread so that after mounting the connector on the female counterpart interlocking is achieved by turning the element.

To enable the attending physician to monitor his or her activities on screen the components of the set of instruments should be designed such that they can be visualized by image-forming methods. Primarily, such image-forming methods are magnetic resonance imaging and radiography. Image forming may be achieved by arranging for the individual components to be manufactured of materials that can be recognized on MR images or radiographs. For example, radiopaque markers made of platinum may be used for this purpose.

The feeding unit is expediently provided with a feed element enabling the compressed implant to be moved in distal direction through the implant needle and finally releasing it in the cyst neck. This feed element, for instance, may have the form of a thin pushrod by means of which the compressed implant can be moved.

Further elucidation of the invention is provided through the enclosed figures by way of examples, where

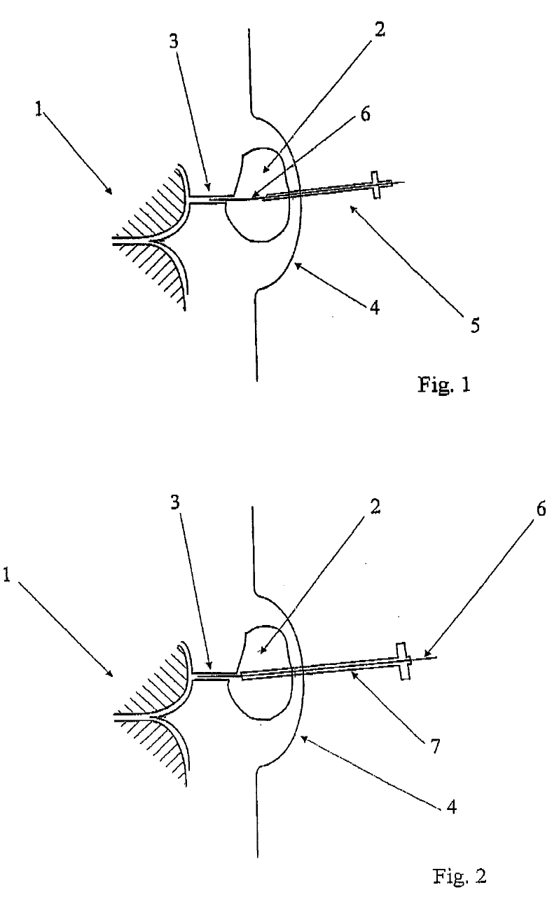

FIG. 1 shows the introduction of guide wire into the cyst neck;

FIG. 2 shows the replacement of the puncture needle by an implant needle;

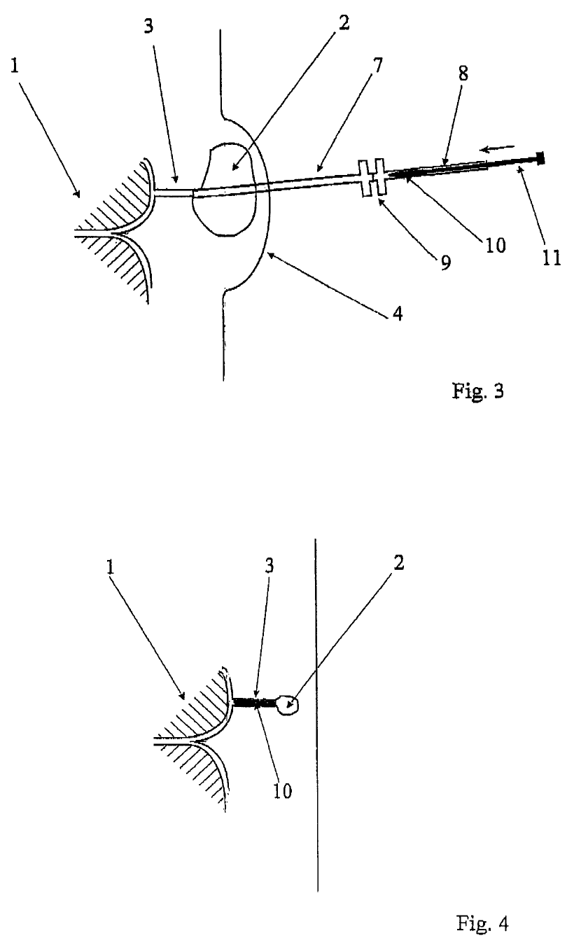

FIG. 3 shows the introduction of an implant into the cyst neck; and

FIG. 4 illustrates the Baker's cyst after successful treatment with the cyst neck obstructed by the implant.

FIG. 1 is a cross-sectional view of a knee where due to a Baker's cyst 2 a sac-like swelling 4 has formed in the hollow of the knee. Via the cyst neck 3 a connection is made up between the Baker's cyst and the knee joint 1 so that fluid from the joint is allowed to penetrate into the tissue and thus causes the Baker's cyst to be filled with joint fluid.

For the treatment the so-called Seldinger technique is applied. For this purpose, at first the Baker's cyst 2 is punctured with the aid of the puncture needle 5. Following this, the guide wire 6 is inserted through the interior of the hollow puncture needle 5 so that the distal end projects into the cyst neck 3. The procedure is monitored with the help of image-forming methods, for example by MR imaging.

As illustrated in FIG. 2 the initially inserted puncture needle 5 is replaced by an implant needle 7 which has a larger diameter. For this purpose, the puncture needle 5 is removed first following which the implant needle 7 is passed over the guide wire 6 until the distal end of the implant needle 7 is adjacent to the entry of the cyst neck 3. At the proximal end of the implant needle 7 there is a connection element via which the feeding unit 8 can be attached to the implant needle 7.

The next treatment step is depicted in FIG. 3. The guide wire 6 which is no longer needed now has been removed and a feeding unit 8 connected to the implant needle 7 via a Luer-Lock connecting element 9. Inside the feeding unit 8 the compressed implant 10 can be seen, with said implant being moved distally with the help of the feeding element 11 and in the direction indicated by arrow from the feeding unit 8 through the implant needle 7 and into the cyst neck 3. As soon as the implant 10 has been introduced successfully into the cyst neck 3 both the feeding unit 8 and the implant needle 7 can be removed and the treatment is completed.

The situation after treatment has been successfully completed can be seen from FIG. 4 which shows that the cyst neck 3 has entirely been filled by the implant 10 preventing joint fluid from flowing from the knee joint 1 towards the Baker's cyst 2. Due to this, the Bakers cyst 2 has become considerably smaller and the sac-like swelling in the hollow of the knee has involuted. First trials have shown that the relapse rate has significantly improved in comparison with methods known hitherto.

1. Use of a spongy material for the production of an implant designed to obstruct a cyst neck.

2. Use according to claim 1, characterized in that the spongy material is a polyvinyl alcohol, polyvinyl acetal, polyacrylate, polyurethane, polystyrene, polyvinyl oxide, polyvinyl fluoride, polyvinyl imidazole, polyethylene oxide, polytetrafluoroethylene, an acyl-substituted cellulose acetate, a chlorine-sulfonated polyolefin or nylon.

3. Use according to claim 2, characterized in that the spongy material is a polyvinyl alcohol.

4. Use according to claim 3, characterized in that the spongy material is Ivanol.

5. Use according to claim 1, characterized in that the implant serves for the occlusion of the neck of a Baker's cyst.

6. Use according to claim 1, characterized in that implant has a cylindrical shape.

7. Use according to claim 1, characterized in that implant has a cuboidal shape.

8. Use according to claim 1, characterized in that implant 3 has an ellipsoidal shape.

9. Use according to claim 6, characterized in that the implant is provided with a thickening element at least one of its longitudinal ends.

10. Use according to claim 1, characterized in that the cross section of the implant amounts to 2 to 7 mm in expanded state.

11. Use according to claim 10, characterized in that the cross section of the implant amounts to approx. 5 mm in expanded state.

12. Use according to claim 1, characterized in that the implant is provided with a coating that enables a visualization by means of magnetic resonance or x-rays.

13. Use according to claim 12, characterized in that the implant is coated with iron, palladium or platinum particles.

14. Set of instruments for the introduction of an implant into a cyst neck, consisting of
a puncture needle for the insertion of a guide wire,
a guide wire, the distal end of which can be inserted into the cyst neck,
an implant needle with connection element arranged at the proximal end,
a feeding unit that can be connected to the implant needle via the connection element, and
an implant 3 of a spongy material that can be introduced into the cyst neck via the feeding unit through the implant needle.

15. Set of instruments according to claim 14, characterized in that said set of instruments also includes a dilatator.

16. Set of Instruments according to claim 14, characterized in that the connection element is a Luer-Lock connector.

17. Set of instruments according to claim 14, characterized in that the components of the set of instruments can be visualized by means of image-forming methods, especially magnetic resonance or x-rays.

18. Set of instruments according to claim 14, characterized in that the feeding unit includes a feed element by means of which the implant can be advanced through the implant needle and released in the cyst neck.

19. Use according to claim 7, characterized in that the implant is provided with a thickening element at least one of its longitudinal ends.

20. Use according to claim 8, characterized in that the implant is provided with a thickening element at least one of its longitudinal ends.
